(19) United States
(12) Patent Application Publication
(10) Pub. No.: US 2015/0313956 A1
(43) Pub. Date: Nov. 5, 2015

(54) COMPOSITIONS AND METHODS FOR HAIR GROWTH

(71) Applicant: NAPIER CONSULTING LLC, Surprise, AZ (US)

(72) Inventor: Candace Napier, Surprise, AZ (US)

(21) Appl. No.: 14/270,307

(22) Filed: May 5, 2014

(51) Int. Cl. A61K 36/71; A61K 31/565; A61K 31/198; A61K 31/573

(52) U.S. Cl. CPC A61K 36/71; A61K 31/573; A61K 31/565; A61K 31/198

(57) ABSTRACT

Various embodiments provide compositions for a hair loss treatment composition for promoting hair growth and/or hair thickness and methods for their manufacture. In various exemplary embodiments, the active agent may comprise a preparation of a steroid, a steroid precursor, an estrogen biomimetic, serotonin agonist, and/or an amino acid. In various embodiments, the active agent may comprise a preparation of at least part of a whole plant, a crude plant extract, an at least partially refined plant extract, and a derivative of the plant extract. Exemplary methods for producing the active agent may comprise combining effective amounts of the components of the active agent. The active agent may be for topical use and may be formulated for delivery to at least one of: a hair follicle, a hair follicle shaft, and skin surrounding the hair follicle.

### BACKGROUND

Hair growth in mammals may vary according to a variety of intrinsic or environmental influences. Intrinsic influences on hair may comprise factors such as an individual's genetic phenotype for hair, age, hormonal changes, nutrition, pregnancy, skin disease, and the like. Environmental influences that may change the rate of hair growth or may decrease thickness may comprise ultraviolet radiation, chemotherapy or other medications.

The hair follicle, the hair shaft-producing mini-organ, comprised of regenerating dermal and epidermal tissue components, periodically undergoes spontaneously repetitive cycles of active hair shaft growth (anagen), follicular regression (catagen), quiescence (telogen), and shedding (exogen). Hair follicle cycling may be modulated by a set of dynamic biochemical, metabolic, and/or immunological interactions. Disruption of hair follicle cycling by intrinsic and environmental influences may result in pathological conditions and diseases of the hair. A curative therapy has not yet been developed.

### SUMMARY

Various embodiments provide compositions for a hair loss treatment composition for promoting hair growth and/or hair thickness and methods for their manufacture. In various exemplary embodiments, the active agent may comprise a preparation of a steroid, a steroid precursor, an estrogen biomimetic, serotonin agonist, and/or an amino acid. In various embodiments, the active agent may comprise a preparation of at least part of a plant, a crude plant extract, an at least partially refined plant extract, and/or a derivative of the plant extract. Exemplary methods for producing the active agent may comprise combining effective amounts of the components of the active agent. The active agent may be for topical use and may be formulated for delivery to at least one of: a hair follicle, a hair follicle shaft, and skin surrounding the hair follicle.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures.

Elements and steps in the figures are illustrated for simplicity and clarity and have not necessarily been rendered according to any particular sequence or scale. For example, steps that may be performed concurrently or in different order are illustrated in the figures to help to improve understanding of embodiments of the present invention.

The figures described are for illustration purposes only and are not intended to limit the scope of the present disclosure in any way. Various aspects of the present invention may be more fully understood from the detailed description and the accompanying drawing figures, wherein:

FIGS. 1A-1B representatively illustrate a photograph of the vertex of a first study participant before and after using an exemplary hair loss treatment composition for six months, respectively;

FIGS. 2A-2B representatively illustrate a photograph of the vertex of a second study participant before and after using an exemplary hair loss treatment composition for six months, respectively;

FIGS. 3A-3B representatively illustrate a photograph of the vertex of a third study participant before and after using an exemplary hair loss treatment composition for six months, respectively; and

FIGS. 4A-4B representatively illustrate a photograph of the vertex of a fourth study participant before and after using an exemplary hair loss treatment composition for six months, respectively.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, methods and systems according to various aspects of the present technology may be practiced in conjunction with any number of systems and methods for producing cosmetic, personal care, and/or pharmaceutical formulations for modulating hair growth and/or hair thickness, and the systems described are merely some exemplary applications for the technology. Various representative implementations may be implemented into any personal care product for topical use on the skin and/or hair of an animal.

The particular implementations shown and described are illustrative of the technology and its best mode and are not intended to otherwise limit the scope of the present technology in any way. For the sake of brevity, conventional manufacturing, processing, preparation, sterilization, and other functional aspects of the system may not be described in detail. Various aspects of the invention provide methods and systems for making and using a topical hair loss treatment composition. A detailed description of various embodiments is provided as a specific enabling disclosure that may be generalized to any application of the disclosed systems and methods in accordance with the various described embodiments.

Exemplary embodiments of the present technology provide a hair loss treat composition comprising one or more active agents that promote at least one of hair growth and/or hair thickness on an animal such as a human. In various embodiments, the active agents may be in the form of a topical composition for application to a target anatomical site, such as hair follicles, hair shafts, and/or surrounding skin.

In various embodiments, the active agent may function to modulate and/or alter any one or more of the rate of the proliferation and/or the differentiation of the hair follicle cells, the rate and/or duration of the hair shaft growth, the hair follicle density (number of hairs/c²), and/or the hair shaft diameter (μm). In some embodiments, the active agent may modulate, induce, inhibit and/or otherwise regulate one or more endogenous hair follicle cycle modulators involved in hair follicle cycling. For example, the active agent may modulate hair growth and/or hair thickness by inducing, inhibiting and/or altering the function of endogenous hair follicle cycle modulators involved in regulating and/or inducing telogen to anagen conversion.

In some embodiments, the active agent may be adapted to modulate hair thickness by increasing the hair shaft diameter, such as by decreasing and/or inhibiting progressive follicular miniaturization of the hair follicle. In various embodiments, the active agent may be adapted to alter hair shaft diameter without modulating the hair follicle cycle. For example, the active agent may comprise a thickening and/or volumizing compound and/or a volumizing substance adapted to alter hair shaft diameter to promote the appearance of hair thickness.

In various exemplary embodiments, the hair loss treatment composition of the present invention may comprise a mixture of active agents. In some embodiments, the hair loss treatment composition may comprise an estrogen biomimetic, a steroid, a steroid precursor, a serotonin agonist, and/or an amino acid.

In various exemplary embodiments, the active agent of the hair loss treatment composition of the present technology may comprise any suitable an estrogen biomimetic compound having an estrogenic effect on the hair follicle that may promote hair growth and/or increase hair thickness. For example, the estrogen biomimetic may comprise a compound that mimics a structural, chemical, physical, and/or functional property of endogenous human estrogen. In some embodiments, the estrogen biomimetic may comprise an endogenous human hormone and/or an exogenous hormone having an estrogenic effect in the human body. In some embodiments, the estrogen biomimetic may comprise a steroid, steroid precursor, and/or active steroid metabolites. For example, in some embodiments, the estrogen biomimetic may comprise at least one of a phytoestrogen, estradiol such as 17-beta-estradiol, estriol, and estrone.

In some embodiments, the phytoestrogen may comprise any one or more of a variety of naturally-occurring plant compounds that may be structurally and/or functionally similar to endogenous mammalian estrogens and/or their active metabolites. For example, the phytoestrogen may comprise lignan compounds found in the plant cell walls of fiber rich plants such as berries, seeds, grains, nuts, and fruits. In some embodiments, the phytoestrogen may also comprise polyphenols, flavonoids, and/or isoflavones.

The phytoestrogen, in accordance with various exemplary embodiments, may be a crude and/or an at least partially refined preparation from a plant. For example, the phytoestrogen may comprise a preparation of at least part of the plant, such as a root or stem that may be ground or pulverized. In some embodiments, the phytoestrogen may comprise a crude plant extract and/or an at least partially refined plant extract wherein one or more biological components of the plant are isolated and/or concentrated. Extraction methodology may comprise any suitable process for isolating and/or concentrating a biological component from a plant such as solvent extraction with one or more polar solvents, non-polar solvents, water, ethanol, chloroform, ethyl acetate, methanol, and the like. In some embodiments, the phytoestrogen may comprise any suitable derivative of the plant extract, such as a product of hydrolysis of the plant extract.

In various embodiments, the preparation of the at least part of the plant, the crude extract, and/or the at least partially refined plant extract may be from a variety of plant sources such as legumes, seeds, nuts, herbs, fruits, vegetables, and grains. For example, plant sources of phytoestrogens may include soy beans and flax seeds. In one embodiment, the phytoestrogen may be contained in a plant from the family Berberidaceae such as Caulophyllum thalictroides, which may be referred to as “blue cohosh.” In another embodiment, the phytoestrogen may be a crude preparation or extract of a plant from the family Apiaceae such as Angelica sinensis, which may be referred to as “dong quoi.”

In one embodiment, the plant comprising the phytoestrogen may be from the buttercup family Ranunculaceae comprising the species Actaea racemosa which may be referred to as “black cohosh.” In some embodiments, a preparation containing the black cohosh may be prepared by grinding, mashing, shredding, or otherwise processing a portion or the whole black cohosh plant. For example, the root or the rhizome of black cohosh may be shredded in a conventional blender. In some embodiments, preparations of black cohosh may comprise a solvent extract of a portion or the whole black cohosh plant, such as isopropanolic and/or ethanolic extracts of the rhizome and/or roots. In addition to phytoestrogens, black cohosh may also comprise other active agents such as saponins (for example, actein and/or cimicifugoside), resins (such as cimicifugin), fukinolic acid, caffeic, and/or isoferulic acid. In various embodiments, the active agent may comprise about 3 mg to about 180 mg of black cohosh.

In various exemplary embodiments, the active agent of the hair loss treatment composition of the present technology may comprise one or more compounds that may mimic and/or modulate the activity of serotonin. For example, the compounds may function as an agonist or antagonist of serotonin in serotonin receptors that may at least partially modulate the hair follicle growth cycle. In one embodiment, the compound may comprise a serotonin agonist. In some embodiments, the serotonin agonist may be a crude plant extract, an at least partially refined preparation from a plant, and or a derivative of the plant extract as explained above.

In one embodiment, the serotonin agonist may comprise a tryptamine alkaloid. For example, the tryptamine alkaloid may comprise N-methylserotonin. N-methylserotonin is a derivative of serotonin with a methyl group located at the alkyl amine. Tryptamine alkaloids such as N-methylserotonin may bind to one or more serotonin receptors as an at least partial serotonin agonist. In some embodiments, black cohosh and/or its extracts or derivatives may comprise the tryptamine alkaloids and/or other compounds that may mimic the activity of serotonin.

In various embodiments of the present technology, the active agent of the hair loss treatment composition may comprise an endogenous steroid hormone such as dehydroepiandrosterone (hereinafter referred to as “DHEA”). DHEA is produced in human adrenal glands, gonads, and the brain and may function as a metabolic intermediary in the biosynthesis of androgen and estrogen. In some embodiments, the source of DHEA and/or precursor compounds to DHEA may comprise a crude plant extract, an at least partially refined preparation from a plant, and or a derivative of the plant extract as explained above. In one embodiment, the source of DHEA and/or precursor compounds to DHEA may be from the plant Dioscorea villosa from the family Dioscoreaceae, commonly referred to as “wild yarn.” For example, the extract may comprise an extract of the rhizomes of the wild yarn. In some embodiments, the wild yarn may comprise glycosides, steroidal saponins, diosgenin, alkaloids, tannins, phytosterols, and starches. In one embodiment, the tubors of the yarn may be extracted with water and/or alcohol to obtain the endogenous steroid hormone. In various embodiments, the active agent may comprise about 3 mg to about 180 mg of Dioscorea villosa.

In various embodiments of the present technology, the hair loss treatment composition may comprise one or more amino acid. The amino acid may be provided in any suitable composition such as a substantially pure composition, a mixture of amino acids that contain the one or more desired amino acid, a preparation of a polypeptide comprising the amino acid, and/or a crude preparation or extract of a plant as a natural source of the amino acid.

In one embodiment, the amino acid may be arginine such as L-arginine. L-arginine may be converted into nitric oxide in the body. Nitric oxide may function as a gasotransmitter in the scalp endothelium that may promote vasodilation of the blood vessels supplying hair follicle cells, resulting in increased blow flow and increased oxygen available to the hair follicles. In one embodiment, L-arginine may promote thickening of the hair follicle resulting in improved volume of the hair. In various embodiments, the active agent may comprise about 3 mg to about 180 mg of L-arginine.

In various embodiments of the present technology, the hair loss treatment composition may comprise non-amino acid vasodilators. For example, in one embodiment, the hair loss treatment composition may comprise 6-Piperidin-1-ylpyrimidine-2,4-diamine 3-oxide, which may be referred to as minoxidil. Minoxidil may function as a nitric oxide agonist and may open potassium channels to hyperpolarize cell membranes. Minoxidil may promote hair growth in the central (vertex) portion of the scalp.

In various embodiments, the hair loss treatment composition may comprise an anti-inflammatory agent. The anti-inflammatory agent may comprise any suitable compound or substance that may modulate and/or decrease inflammation of hair follicles and/or the surrounding skin. For example, in various embodiments, the anti-inflammatory agent may comprise at least one of corticosteroid such as hydrocortisone, fluocinonide (for example, the drug known as Lidex®), betametasone, dexmethasone, and triamicinolone.

In various embodiments, the hair loss treatment composition may be combined with any suitable functional additive to enhance the percutaneous absorption or penetration of the hair loss treatment composition into the hair follicle, maintain the stability of the components of the hair loss treatment composition, and/or to support the function of the hair loss treatment composition to grow hair. In various embodiments, the functional additive may comprise at least one of an emulsifier, stabilizer, solubilizer, detergent, solvent, antioxidant, buffer, emollient, humectant, occlusive agent, and preservative. For example, the functional additive may comprise purified water, propylene glycol, ethyl alcohol such as denatured 200 proof ethyl alcohol, dimethylsulfoxide, a vitamin, and an acidifier. In some embodiments, the functional additive may provide an aesthetically desirable property such as a fragrance, a dye, and/or a pigment.

In various embodiments, the hair loss treatment composition may be implemented into a topical delivery medium formulated to deliver the hair loss treatment composition to the skin and/or to the hair follicles. The topical delivery medium may comprise any substance suitable for topical application to the hair and/or the skin for delivering the hair loss treatment composition. In some embodiments, the topical delivery medium may facilitate transdermal and/or transfollicular delivery of the hair loss treatment composition. In various embodiments, the topical delivery medium may be adapted to selectively deliver the hair loss treatment composition to the hair follicle cells while limiting substantial delivery of the hair loss treatment composition to cells external to the hair follicle cells. For example, in some embodiments, the topical delivery medium may comprise liposomes adapted to penetrate the stratum corneum. In various embodiments, the topical delivery medium may comprise one or more of a gel, scrub, soap, oil, sunscreen, ointment, cream, lotion, solution, spray, foam, tonic, conditioner, shampoo, and the like.

In accordance with various exemplary embodiments, including the Example formulations listed below, methods for preparing the hair loss treatment compositions may comprise shredding a whole black cohosh plant such as using a blender. The indicated weight of the shredded black cohosh plant may be combined with any other components of the active agent, the functional additives, and/or the topical delivery medium and heated. In embodiments comprising alcohol, the alcohol may be added last. In one embodiment, the temperature may be brought to about 75° C. followed by inactivation of the heating element or removal of the heat source. The application of heat may effect mixing of the components of the active agent by at least partially solubilizing the shredded black cohosh within the other components. In some embodiments, the hair loss treatment composition may be sufficiently mixed upon appearing to be homogenous with a pale tan color.

Example 1 illustrates an approximate composition of a hair loss treatment composition having a final volume of about 60 ml:

### EXAMPLE 1

| | | |
|---|---|---|
| Minoxidil 5.0 w/v % | 300 | mg |
| Black Cohosh | 120 | mg |
| Hydrocortisone (0.25 mg/mL) | 15 | mg |
| Purified Water up to | 60 | mL |
| Total | 60 | mL |

Example 2 illustrates an approximate composition of a hair loss treatment composition having a final volume of about 60 ml:

### EXAMPLE 2

| | | |
|---|---|---|
| Minoxidil 5.0 w/v % | 300 | mg |
| Estradiol | 10 | mg |
| Purified Water up to | 60 | mL |
| Total | 60 | mL |

Example 3 illustrates an approximate composition of a hair loss treatment composition having a final volume of about 60 ml:

### EXAMPLE 3

| | | |
|---|---|---|
| Minoxidil 5.0 w/v % | 300 | mg |
| Black Cohosh | 120 | mg |
| Hydrocortisone (0.25 mg/mL) | 15 | mg |
| Purified Water up to | 60 | mL |
| Total | 60 | mL |

Example 4 illustrates an approximate composition of a hair loss treatment composition having a final volume of about 60 ml:

### EXAMPLE 4

| | | |
|---|---|---|
| Minoxidil 5.0 w/v % | 300 | mg |
| Estradiol | 2 | mg |
| Hydrocortisone (0.25 mg/mL) | 15 | mg |
| Purified Water up to | 60 | mL |
| Total | 60 | mL |

Example 5 illustrates an approximate composition of a hair loss treatment composition having a final volume of 60 mL:

### EXAMPLE 5

| | | |
|---|---|---|
| DHEA 3.0 w/v % | 180 | mg |
| Black Cohosh | 180 | mg |
| L-arginine | 180 | mg |
| Purified Water up to | 60 | mL |
| Total | 60 | mL |

Example 6 illustrates an approximate composition of a hair loss treatment composition having a final volume of about 60 mL:

### EXAMPLE 6

| | | |
|---|---|---|
| Black Cohosh | 180 | mg |
| Wild Yam | 180 | mg |
| L-arginine | 180 | mg |
| Propylene Glycol | 12 | mL |
| Ethyl Alcohol 200 Proof | 32 | mL |
| Purified Water up to | 60 | mL |
| Total | 60 | mL |

Example 7 illustrates an approximate composition of a hair loss treatment composition having a final volume of about 60 mL:

### EXAMPLE 7

| | | |
|---|---|---|
| Black Cohosh | 3 | mg |
| Wild Yam | 3 | mg |
| L-arginine | 3 | mg |
| Propylene Glycol | 10 | mL |
| Ethyl Alcohol 200 proof | 34 | mL |
| Purified Water up to | 60 | mL |
| Total | 60 | mL |

Example 8 illustrates an approximate composition of a hair loss treatment composition having a final volume of 60 mL:

### EXAMPLE 8

| | | |
|---|---|---|
| Minoxidil 5.0 w/v % | 300 | mg |
| Black Cohosh | 120 | mg |
| Propylene Glycol | 30 | mL |
| Ethyl Alcohol | 18 | mL |
| Hydrocortisone (0.25 mg/mL) | 15 | mg |
| Purified Water up to | 60 | mL |
| Total | 60 | mL |

The hair loss treatment composition of Example 6 was tested on four female study participants over a period of six months. Each study participant applied the composition of Example 6 to the scalp skin using a liquid dropper and a dragging motion across the scalp, followed by massaging the composition into the scalp with the fingertips. At the time of application, the hair was either dry or towel dried after washing. The composition was allowed to soak into the scalp for a four minute period before the application of any additional personal care products were applied to the scalp and/or hair.

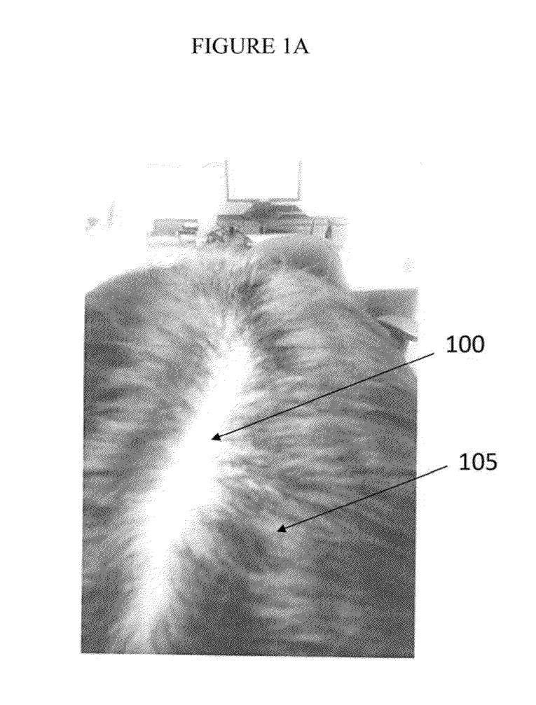

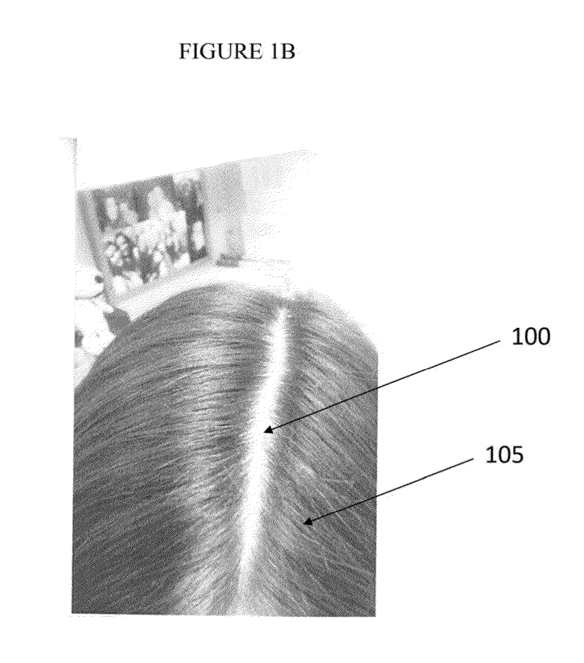

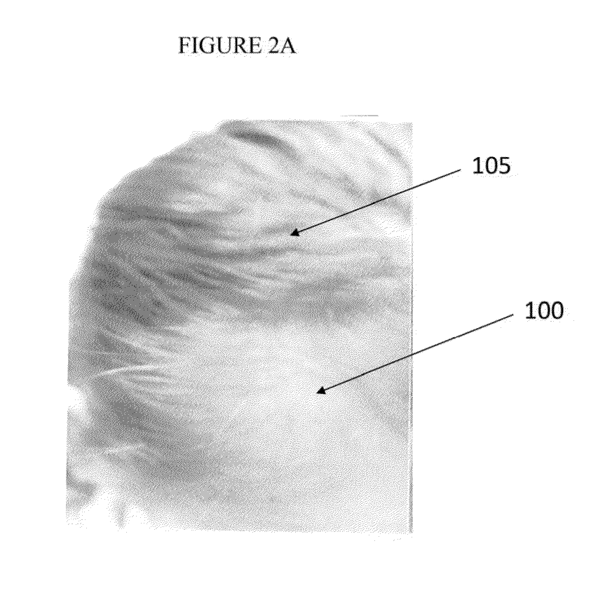

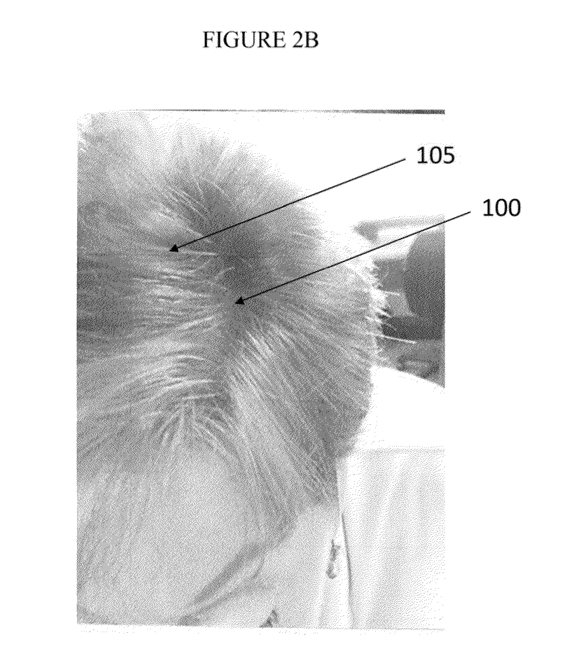

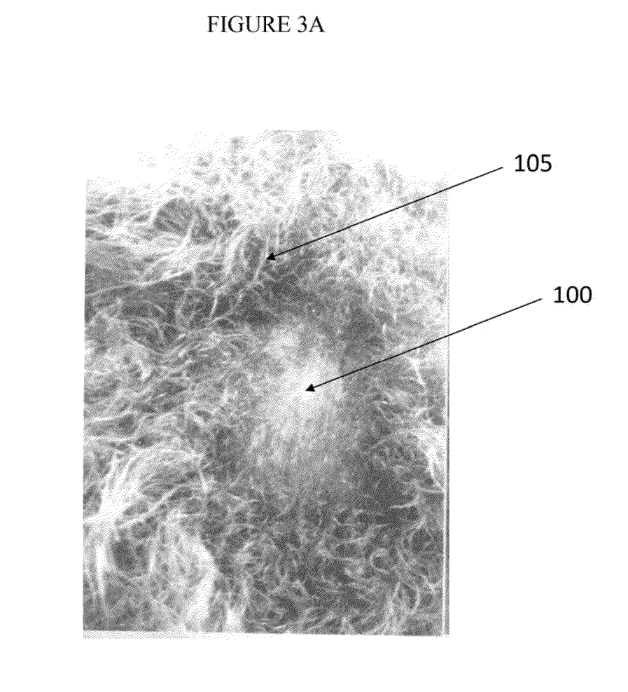

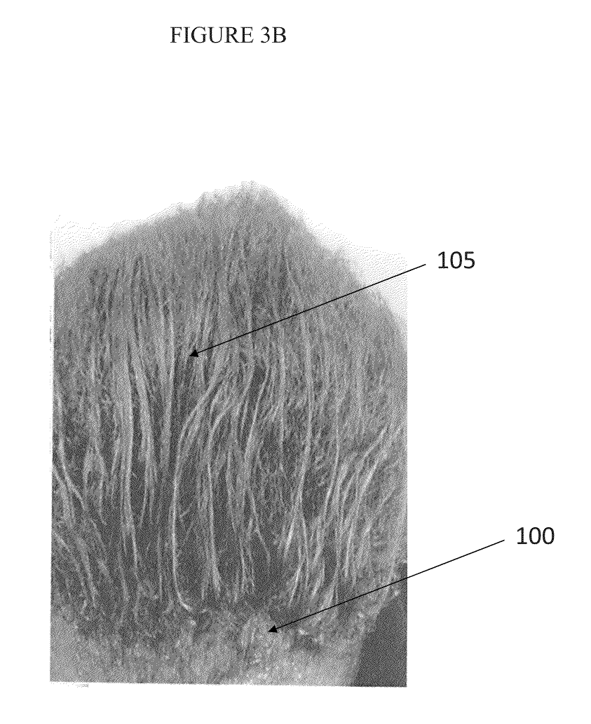

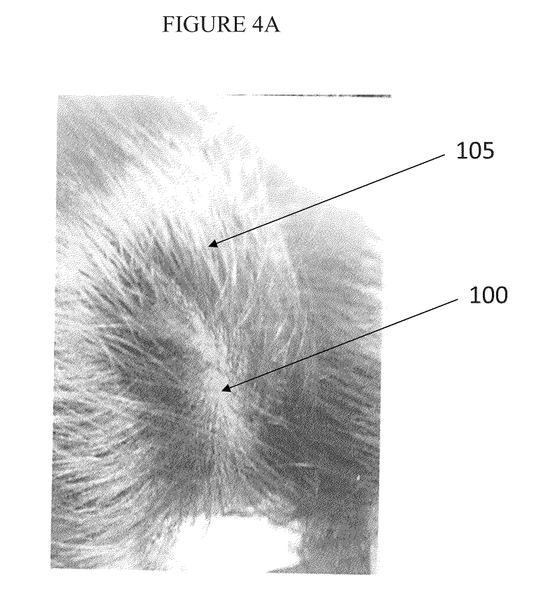

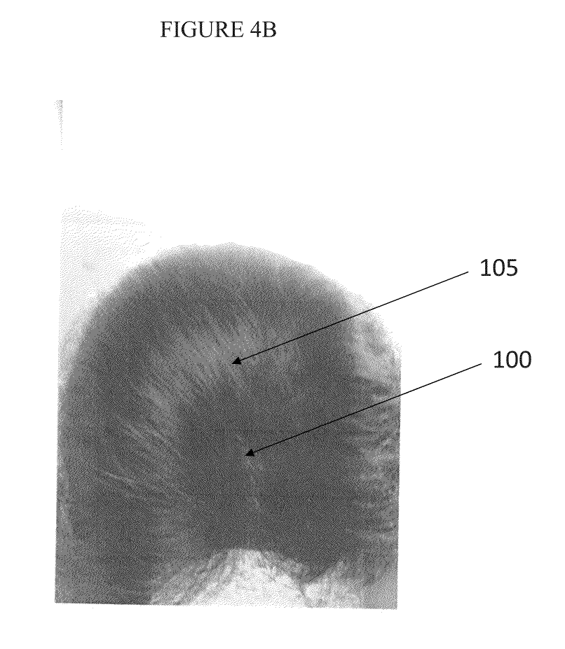

Gross visual analysis of the visible hair and visible skin was documented by photography and illustrated in FIGS. 1-4 wherein the vertex of the scalp of each subject is represented in each Figure. Referring to FIGS. 1A, 2A, 3A and 4A, the skin 100 is abundantly visible under the hair 105 due to thinning and absence of the hair 105 before treatment, as shown by the decreased density of the hair 105. Referring FIGS. 1B, 2B, 3B and 4B, after six months of treatment, the hair 105 exhibits markedly increased density and significantly less skin 100 is visible.

In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition, system, or apparatus that comprises a list of elements does not include only those elements recited, but may also include other elements not expressly listed or inherent to such process, method, article, composition, system, or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

What is claimed is:

1. A hair loss treatment composition for promoting the growth of hair on an animal, comprising:
an active agent, comprising:
at least one of a steroid and a steroid precursor;
at least one of an estrogen biomimetic and a serotonin agonist; and
an amino acid;
wherein the application of the active agent to the hair on the animal increases the growth of the hair relative to the growth of the hair without the active agent.

2. The composition of claim 1, wherein the at least one of the estrogen biomimetic and the serotonin agonist comprises a tryptamine alkaloid.

3. The composition of claim 1, wherein the at least one of the estrogen biomimetic and the serotonin agonist comprises a preparation from a plant.

4. The composition of claim 3, wherein the preparation from the plant comprises at least one of a portion of the whole plant, a crude plant extract, an at least partially refined plant extract, a derivative of the crude plant extract, and a derivative of the at least partially refined plant extract.

5. The composition of claim 4, wherein the plant is from the family Ranunculaceae.

6. The composition of claim 5, wherein the plant from the family Ranunculaceae is Actaea racemosa.

7. The composition of claim 6, wherein the active agent comprises about 3 mg to about 180 mg of the preparation from the plant.

8. The composition of claim 4, wherein the plant is from the family Berberidaceae.

9. The composition of claim 8, wherein the plant from the family Berberidaceae is Caulophyllum thalictroides.

10. The composition of claim 4, wherein the plant is from the family Apiaceae.

11. The composition of claim 10, wherein the plant from the family Apiaceae is Angelica sinensis.

12. The composition of claim 1, wherein the at least one of the estrogen biomimetic and the serotonin agonist comprises a phytoestrogen.

13. The composition of claim 1, wherein the at least one of the estrogen biomimetic and the serotonin agonist comprises at least one of estradiol, estriol, and estrone.

14. The composition of claim 1, wherein a source of the at least one of the steroid and the steroid precursor comprises a preparation from a plant.

15. The composition of claim 14, wherein the preparation from the plant comprises at least one of a portion of the whole plant, a crude plant extract, an at least partially refined plant extract, a derivative of the crude plant extract, and a derivative of the at least partially refined plant extract.

16. The composition of claim 15, wherein the plant is from the family Dioscoreaceae.

17. The composition of claim 16, wherein the plant from the family Dioscoreaceae is a Dioscorea spp.

18. The composition of claim 17, wherein the active agent comprises about 3 mg to about 180 mg of the preparation from the plant.

19. The composition of claim 1, wherein the at least one of the steroid and the steroid precursor comprises at least one of dehydroepiandrosterone and a corticosteroid.

20. The composition of claim 1, wherein the amino acid comprises L-arginine.

21. The composition of claim 20, wherein the active agent comprises about 3 mg to about 180 mg of the L-arginine.

22. The composition of claim 1, wherein the hair loss treatment composition further comprises a topical delivery medium.

23. The composition of claim 22, wherein the topical delivery medium comprises at least one of a gel, scrub, ointment, cream, lotion, solution, spray, foam, oil, conditioner, tonic, soap, sunscreen, liposome, and shampoo.

24. The composition of claim 1, wherein the hair loss treatment composition further comprises a functional additive.

25. The composition of claim 24, wherein the functional additive is at least one of an antioxidant, fragrance, dye, pigment, preservative, emollient, humectant, occlusive agent, emulsifier, stabilizer, solubilizer, detergent, buffer, water, propylene glycol, alcohol, solvent, vitamin, and acidifier.

26. A hair loss treatment composition for promoting the growth of hair on an animal, comprising:
an active agent, comprising:
a preparation of a Dioscorea spp. of plant;
a preparation of a Actaea racemosa plant; and
L-arginine;
wherein the application of the active agent to the hair on the animal increases the growth of the hair relative to the growth of the hair without the active agent.

27. The hair loss treatment composition of claim 26, wherein active agent comprises about 3 mg to about 180 mg of each of the preparation of the Dioscorea spp. of plant, the preparation of the Actaea racemosa plant, and the L-arginine.

28. The hair loss treatment composition of claim 26, further comprising propylene glycol and ethyl alcohol.

29. A method for promoting the growth of hair in hair follicles on an animal, comprising:
preparing a hair loss treatment composition comprising an active agent, wherein the active agent is prepared by mixing:
at least one of a steroid and a steroid precursor;
at least one of an estrogen biomimetic and a serotonin agonist; and
an amino acid;
wherein the active agent at least partial solubilized by heating.

30. The method of claim 29, further comprising delivering an effective amount of the hair loss treatment composition to the hair follicles.

31. The method of claim 29, further comprising mixing the active agent with a functional additive comprising at least one of an antioxidant, fragrance, dye, pigment, preservative, emollient, humectant, occlusive agent, emulsifier, stabilizer, solubilizer, detergent, buffer, water, propylene glycol, alcohol, solvent, vitamin, and acidifier.

32. The method of claim 29, further comprising mixing the active agent with a topical delivery medium comprising at least one of a gel, scrub, ointment, cream, lotion, solution, spray, foam, oil, conditioner, tonic, soap, sunscreen, liposome, and shampoo.

33. The method of claim 29, wherein the at least one of the steroid and the steroid precursor comprises a preparation of at least one of: a portion of the whole plant, a crude plant extract, an at least partially refined plant extract, a derivative of the crude plant extract, and a derivative of the at least partially refined plant extract from the family Dioscoreaceae.

34. The method of claim 29, wherein the at least one of the steroid and the steroid precursor comprises at least one of dehydroepiandrosterone and a corticosteroid.

35. The method of claim 29, wherein the amino acid comprises L-arginine.

36. The method of claim 29, wherein the at least one of the estrogen biomimetic and the serotonin agonist is at least one of a portion of the whole plant, a crude plant extract, an at least partially refined plant extract, a derivative of the crude plant extract, and a derivative of the at least partially refined plant extract from the family Berberidaceae.

37. The method of claim 29, wherein the at least one of the estrogen biomimetic and the serotonin agonist is at least one of phytoestrogen, estradiol, estriol, and estrone.
